(19) United States
(12) Patent Application Publication
(10) Pub. No.: US 2006/0068022 A1
(43) Pub. Date: Mar. 30, 2006

(54) Bioactive agent compositions for repair of cell injuries

(76) Inventor: Raymond J. Playford, London (GB)

Correspondence Address: MERCHANT & GOULD PC, P.O. BOX 2903, MINNEAPOLIS, MN, 55402-0903, US

(21) Appl. No.: 10/953,244

(22) Filed: Sep. 29, 2004

(51) Int. Cl. A61K 35/20; A61K 33/00; A61K 36/484; A61K 36/48

(52) U.S. Cl. 424/535; 424/757; 424/717

(57) ABSTRACT

A method for prophylactically treating a gastrointestinal disorder or skin ailment in a mammal through the administration of an enhanced bioactive agent composition comprising a bioactive agent and at least one of a soy product, licorice product, or sodium bicarbonate (depending upon the indication to be treated) is provided according to the invention. The bioactive agent preferably is bovine colostrum. The effective amount of enhanced bioactive agent composition to be used will depend upon such factors as the age and weight of the mammal, the bioactivity level of the bioactive agent, the gastrointestinal disorder or skin ailment at issue, and whether treatment of existing symptoms of the gastrointestinal disorder or skin ailment, or prevention of the onset of such symptoms is desired. A medicament comprising an enhanced bioactive agent composition for prophylactically treating a gastrointestinal disorder or skin ailment in a mammal is also provided according to the invention.

### FIELD OF INVENTION

This invention relates to a method for using a bioactive agent composition synergistically enhanced with soy, licorice, and/or sodium bicarbonate additives to prophylactically prevent and/or treat cellular injuries in the gastrointestinal tract or skin of mammals, and compositions for such treatments.

### BACKGROUND OF THE INVENTION

Organs in humans and animals are composed of tissues, which in turn are composed of cells. Epithelial tissue covers body surfaces and lines body cavities. Endothelial cells perform a similar role in forming a lining layer in tissues such as blood vessels, lymph tissues, and urogential system. Connective tissue cells maintain cohesiveness between cells, and act as a “scaffold” in both normal tissues and as part of the repair cell process. Epithelial, endothelial, and connective tissue cells are therefore important for the defense, normal structure, and repair of mammalian animals, including protecting the internal environment of the body against the external environment, and containing fluids in various organs.

Such organs of the human or animal body include the gastrointestinal system, which in turn includes the stomach and the small and large intestines. Eaten food is collected in the stomach, whereupon it is digested in the duodenum portion of the small intestines, and then passed on to the jejunum and ileum portion of the small intestines where the nutrients (e.g., fatty acids, sugars, amino acids) from the digested food are absorbed. The remaining digesta is then passed along to the large intestine (sometimes called the “bowel” or “gut”) in which fluids from this solid mass of digested food are absorbed. The leftover, used portion of the food enters the rectum for subsequent discharge from the body.

The epithelial tissue lining of the gastrointestinal tract possess the remarkable ability to remain intact despite being constantly bathed in acid and proteolytic enzymes that can digest virtually any form of food that is eaten. When a superficial mucosal injury occurs, such as following direct physical trauma or ingestion of noxious agents like aspirin or alcohol, it is rapidly healed. This healing process is achieved through migration of surviving cells around the wound edge to cover the denuded area of the tissue within the first hour after the injury, followed by differentiation and multiplication (“proliferation”) of the cells beginning one to two days after the injury. Finally, remodeling occurs where the mucosa slowly re-establishes an essentially normal looking mucosa. The intestines therefore possess powerful mucosal defense and repair mechanisms.

A common problem that affects the gastrointestinal system is inflammation of the epithelial cell lining. This arises in the form of esophagitis in the esophagus, which can be caused by acid reflux, and gastritis in the stomach, which can be caused by bacteria like Helicobacter Pylori, or chemicals ingested into the body such as iron supplements and non-steroidal anti-inflammatory drugs (NSAIDs) like aspirin, ibuprofen, flurbiprofen, ketoprofen, and indomethacin. Duodenitis is inflammation of the first part of the small intestine. A much more serious form of inflammation of the small or large intestine is inflammatory bowel disease (“IBD”) characterized by gross inflammation that is out of control, an intestinal lining that bleeds and is ulcerated, and weight loss. Crohn's Disease is a form of IBD. Diverticular disease represents bacterial inflammation of the large intestine, and typically afflicts people who suffer from constipation.

In general, an ulcer is any eroded area of the skin or mucous membrane marked by tissue disintegration. More commonly, however, ulcer is used to refer to disorders in the upper digestive tract. It is estimated that approximately 10% of the United States population will develop an ulcer at some point in their lives. Peptic ulcers can develop in the lower part of the esophagus, the stomach, and the duodenum and jejunum portions of the small intestines. Peptic ulcers are caused by infection by Helicobacter Pylori bacteria, NSAIDs, and disorders like Zollinger-Ellison syndrome that cause over-secretion of stomach juices. Symptoms for such peptic ulcers include heartburn, stomach pain relieved by eating or antacids, weight gain, and a burning sensation at the back of the throat.

Gastric ulcers account for about 25% of peptic ulcers, and are most commonly caused by the use of NSAIDs, or by Helicobacter infection. Symptoms of gastric ulcers include feelings of indigestion and heartburn, weight loss, and repeated episodes of gastrointestinal bleeding.

About 5% of ulcer patients actually develop perforations, which are holes in the duodenal or gastric wall through which the stomach contents can leak out into the abdominal cavity. Emergency surgery may be required to treat such a perforation.

Fortunately, however, most patients with esophagitis and peptic and gastric ulcers can be medicated with drugs to create chemical reactions that either lower the rate of stomach acid secretion, or protect the mucous tissues that line the digestive tract. Such antisecretory drugs include: proton pump inhibitors, which bind an enzyme that secretes stomach acid, like omeprazole (Prilosec®) and lansoprazole (Prevacid®); or H₂ receptor antagonists like ranitidine (Zantac®), cimetidine (Tagamet®), famotidine (Pepcid®), or nizatidine (Axid®). Drugs currently used to protect the stomach tissues include sucralfate (Carafate®), bismuth preparations, and misoprostol (Cytotec®). These drugs act to neutralize disorders like excessive acid secretion that would otherwise interfere with the natural function of cell migration and proliferation to heal the ulcer.

Drug treatment is the primary method used to alleviate symptoms of both ulcerative colitis and Crohn's disease. This includes anti-inflammatory drugs such as mesalamine, olsalazine, and balsalazide, and anti-TNF antibodies. Immunosuppressive agents work to restrain the immune system from attacking the body's own tissues to cause further inflammation. Surgery may be required if a patent does not respond to medicines for treating IBD.

The mouth also forms part of the gastrointestinal tract. One of the most common medical problems with the mouth is apthous ulcers. Apthous ulcers are painful, shallow ulcers on the tongue or mucosal surface of the mouth. The ulcer starts as a small vesicle that subsequently breaks down. Healing occurs without scarring. Apthous ulcers are relatively common, occurring in approximately 20% of the human population—more typically in women than men. The vast majority of apthous ulcers occur as an isolated finding. Much more rarely, they are associated with Crohn's disease, Celiac disease, or Behcet's syndrome.

For the normal apthous ulcer not associated with a specific condition, medical treatment is generally not given, as it is only of limited benefit. For those apthous ulcers that do require treatment, measures that may help in the management of an acute episode mainly involve hydrocortisone lozenges (usually one lozenge four times per day), or adocortyl (Orobase®) applied in a thin layer over the ulcer 2-4 times daily.

There is evidence that regular use of chlorhexidine gluconate, an antibacterial agent, administered as a mouth rinse, increases the number of apthous ulcer-free days, and reduces the severity of each episode of ulceration, compared with control preparations. See Chadwick, B., Addy, M., Walker, D. M. et al., “Hexetidine Mouthrinse in the Management of Minor Apthous Ulceration, and as an Adjunct to Oral Hygiene,” British Dent. J. 171: 83-7 (1991); Meiller, T. F., Kutcher, M. J., Overholser, C. D. et al.,“Effect of an Antimicrobial Mouthrinse on Recurrent Apthous Ulcerations,” Oral. Surg. Oral. Med. Oral. Pathol. 72: 425-29 (1991). However, such mouthrinses do not appear to reduce the incidence of recurrent ulceration.

It has been found, however, that alternative treatments using bioactive agents instead of traditional drug chemicals can effectively treat ulcers by rebalancing the stomach's hydrochloric acid output, and/or enhancing the mucosal lining of the mouth, stomach and intestines through promotion of these natural cell migration and proliferation functions. Such bioactive agents include plant extracts like aloe vera, deglycrrhizinated licorice (DGL) in a chewable or powder form, raw cabbage juice, substances of animal origin, artificially produced nutritional molecules like zinc-carnosine, and artificially made normal proteins like recombinant human spasmolytic polyptide (hSP). One particularly important example of an animal-originated bioactive agent is colostrum, which is the first milk produced after birth, and has been shown to enhance the cell migration and growth healing function of ulcerated digestive tracts caused by NSAIDs. See Playford, R. J., Floyd, D. N., MacDonald, C. E. et al., “Bovine Colostrum in a Health Food Supplement Which Prevents NSAID—Induced Gut Damage,” Gut 44: 653-58 (1999); Playford, R. J., MacDonald, C. E., Calnan, D. P. et al., “Colostrum, Reduces the Acute, Non-Steroidal, Anti-Inflammatory Drug-Induced Increase In Intestinal Permeability,” Clinical Science 100: 627-33 (2001); Playford, R. J., MacDonald, E., Calnan, D. P. et al., “Co-Administration of the Health Food Supplement, Bovine Colostrum, Reduces the Acute Non-Steroidal Anti-Inflammatory Drug-Induced Increase in Intestinal Permeablility,” Clinical Science 100: 627-33 (2001), See also EPO Patent Nos. 927,042 and 936, 917 issued to Johnson and Playford. Similarly, another study has shown that hSP may be used to enhance cell migration in order to reduce gastric damage by 50%. See Playford, R. J., Marchblank, T., Chinery, R. et al., “Human Spasmolytic Polypeptide Is A Cytoprotective Agent That Stimulates Cell Migration,” Gastroenterology 108: 108-16 (1995). Zinc-carnosine manufactured by Lonza Incorporated may also be used to treat stomach ulcers and gastric reflux.

In another study, it has been demonstrated that patients suffering from distal colitis (a form of IBD) showed marked improvement after taking a colostrum enema in combination with 5-aminosalicylic acid mesalazine, compared with the control group receiving mesalazine and a placebo enema. See Khan, Z., MacDonald, C., Wicks, A. C. et al., “Use of the “Nutriceutical, Bovine Colostrum, for the Treatment of Distal Colitis: Results from an Initial Study,” Ailment Pharmacological Therapy 16: 1917-22 (2002). For such IBD symptoms, the growth factors contained in colostrum stimulate the intestinal cells to repair themselves through proliferation and restitution.

Plant sources of bioactive compounds have traditionally been used by herbalists and indigenous healers for the prevention and treatment of peptic ulcer. For example, curcumin capsules improve endoscopic healing of peptic ulcers, as well as improving symptoms of patients with non-ulcer dyspepsia. Other botanical compounds with anti-ulcer activity include flavonoids (i.e. quercetin, naringin, silymarin, anthocyanosides, sophoradin derivatives) saponins (i.e. from Panax japonicus and Kochia scoparia), tannins (i.e. from Linderae umbellatae), gums and mucilages (i.e. gum guar and myrrh). Among herbal drugs, licorice, aloe gel and capsicum have been used extensively and their clinical efficacy documented. Also, ethnomedical systems employ several plant extracts for the treatment of peptic ulcer. Taken together, the evidence suggests that the plant and animal kingdom might provide a useful source of new anti-ulcer compounds for development as pharmaceutical entities or alternatively, as simple dietary adjuncts to existing therapies.

Ghosh, S. and Playford, R. J., “Bioactive Natural Compounds for the Treatment of Gastrointestinal Disorders”, Clinical Science 104, 547-56 (2003), provides a survey of many other bioactive compounds that have been found useful for the treatment of various gastrointestinal and other disorders. For example, curcumin capsules improve endoscopic healing of peptic ulcers, as well as improving symptoms of patients with non-ulcer dyspepsia. Sangre de grado has also been shown to heal experimental gastric ulcers induced by application of 80% acetic acids in rats. Moreover, acemannan, a component of aloe vera, prevents stress-induced gastric ulceration in rats.

A further potential plant source of bioactive compounds is soybeans (Glycine Max). Soybean-based foods are the predominant source of dietary isoflavones, and are also a rich source of trypsin inhibitor, phosphatidyl inositol, saponins, and sphingolipids, all of which have potential health benefits, including cancer prevention, and prevention of ischaemic heart disease and regulation of the host immune system. See Funk. M. A. and Baker, D. H., “Effect of Soy Products on Methotrexate Toxcity in Rats,” Journal of Nutrition 121(10): 1684-92 (October 1991); Wang, W. and Higunchi, C. M., “Dietary Soy Protein is Associated With Reduced Intestinal Mucosal Polyamine Concentration in Male Wistar Rats.” American Society for Nutritional Sciences Manuscript No. 0022-3166100 (2000); Wang. W., Higuchi, C. M., and Zhang, R., “Individual and Combinatory Effects of Soy Isoflavones on the Invitro Potentiation of Lymphocycte Activation,” Nutr. Cancer. 29(1): 29-34 (1997). Soybean protein has also been used as an alternative protein source to cows milk for use in infant formula and other food products, since 15% of U.S. infants are lactose intolerant. A typical preparation is reported by Morales de Leon, J., Graeve, W. R., Villalobos, M. E., and Bourges, R. H., “Development of an Infant Food Product Based on Fermented Milk, Cereals, and Soybeans,” Arch. Latinoam Nutr. 38(4): 852-64 (December 1988).

Some research has also been directed to determine whether there is any link between soybean products and repair of cellular tissues. For example, in one study the addition of low doses of soybean agglutinin or soy protein to intraveneously-fed animals affected by shrunken small intestines seemed to promote some small intestinal growth. See Li, Z; Li, D., and Qiao, S., “Effects of Soybean Agglutinin on Nitrogen Metabolism and on Characteristics of Intestinal Tissues and Pancreas in Rats,” Arch. Tierernahr. 57(5): 369-80 (October 2003). Another study demonstrated that soybean agglutinin binds to corneal (eye) endothelial cells during wound repair, and alters their appearance under the microscope, although it seemed to have no effect on the amount of cell migration that occurred. See Gordon, S. R. and Wood, M., “Soybean (Glycine Max) Agglutinin Binds to Corneal Endothelial Cells During Wound Repair and Alters Their Microfilament Pattern,” Cell Molecular Biology 43(3): 329-36 (May 1997). A cell culture study looking at the effect of a component of soybean, called genistein isoflavone, on gut growth suggested that ingestion of this material might influence gut growth but the effect may vary according to the dose used. Administration of a relatively small amount of genistein isoflavone (1 mg protein/ml) appeared to increase some parameters of growth of Caco2-BBe cells, whereas a 30-fold higher genistein concentration, similar to levels potentially found in soya-based infant formulas, actually reduced cell growth by between 40% and 94%, depending on the assay method used. See Chen, A. C. and Donovan, S. M., “Genistein at a Concentration Present in Soy Infant Formula Inhibits Caco-2BBe Cell Proliferation By Causing G2/M Cell Arrest,” American Society for Nutritional Sciences Manuscript No. 0022-3166/04 (2004). In a placebo-controlled human clinical trial, it was found that soybean milk was not effective in healing gastric ulcers, although it did appear to reduce some of the discomfort associated with peptic ulcers. Fung, W. P., “Effect of Soya Bean Milk on the Healing of Gastric Ulcers—A Controlled Endoscopic Study,” Med. J. Aust. 1(23): 717-18 (1975).

In addition to soybean-based products, another potential source of bioactive compounds is licorice root (Glycyrrhiza radix). Licorice root is one of the oldest and most frequently employed botanicals in Chinese medicine. In the U.S., licorice products are usually used as flavoring and sweetening agents in food products. Constituents of licorice include triterpenoids, such as glycyrrhizin and its aglycone glycyrrhizic acid, various polyphenols, and polysaccharides. Various chemical modifications of glycyrrhizic acid have also been produced such as the salts, amides, and glycopeptides derivatives.

Research suggest that licorice may have a number of pharmaceutical effects, including anti-cancer, anti-viral, and anti-inflammatory effect, as well as facilitating drug delivery through the skin. See, e.g., Wang, Z. Y. and Nixon, D. W., “Licorice and Cancer,” Nutr. Cancer. 39: 1-11 (January 2001); Takahashi, T., Takasuka, N., Iigo, M. et al., “Isoliquiritigenin, a Flavonoid From Licorice, Reduces Prostaglandin E2 and Nitric Oxide, Causes Apoptosis, and Suppresses Aberrant Crypt Foci Development,” Cancer Science 95: 448-53 (May 2004); Armanini, D., DePalo, C. B., Mattrarello, M. J. et al., “Effect of Licorice on the Reduction of Body Fat Mass in Healthy Subjects,” Journal Endocrinol. Invest. 26: 646-50 (July 2003); Nokhodchi, A., Nazemiyeh, H., Ghafourian, T. et al., “The Effect of Glycyrrhizin on the Release Rate and Skin Penetration of Diclofenac Sodium From Topical Formulations,” Pharmacology 57: 883-88 (November 2002); Sasaki, H., Takei, M., Kobayashi, M. et al. “Effect of Glycyrrhizin, An Active Component of Licorice Roots, On HIV Replication in Cultures of Peripheral Blood Mononuclear Cells from HIV-Seropositive Patients,” Pathobiology 70(4): 229-36 (2002-2003); Abe, M., Akbar, F., Hasebe, A. et al., “Glycyrrhizin Enhances Interleukin-10 Production by Liver Dendritic Cells in Mice With Hepatitis,” Journal of Gastroenterology 38(10): 962-67 (2003).

However, trials of licorice derivatives for healing gastric results have exhibited mixed results. In one placebo-controlled clinical trial of 96 patients with gastric ulcers who were treated with deglycyrrhizinized licorice (“DGL”) or a placebo, no differences were detected in the amount of patients with complete or partial healing or in their overall clinical improvement after four weeks of treatment. See Bardhan, K. D., Cumberland, D. C., Dixon, R. A., and Holdsworth, C. D., “Clinical Trial of Deglycyrrhizinized Liquorice in Gastric Ulcer,” Gut 19: 779-82 (September 1978). One synthetically made product from licorice that has reached the clinical arena for treating gastric ulcers is carbenoxalone. This synthetically prepared drug is an ester derivative of glycyrrihizic acid. Clinical trials have shown it to be beneficial for treating gastric ulcers in both humans and rats, and one of its key mechnisms is probably acting through postaglandin formation. Franco, L., Manara, P., Erbetti, I., and Velo, G. P., “Anti-Ulcer Activity of Carbenoxolone and ISF 3401 on PGE2 Release in Rat Gastric Mucosa,” Pharmacological Research 27(2): 141-50 (February-March 1993). One of the unfortunate side effects of carbenoxolone, though, is sodium retention within the body, which causes it to retain water and in severe cases causes oedema (swelling of the legs). This was a concern when high doses of carbenoxalone were used as a drug, but can also be a problem if extremely high doses of licorice-root are consumed. See Olukoga, A. and Donaldson, D., “Liquorice and Its Health Problems,” Journal R. Soc. Health 120(2): 83-9 (June 2000); Borelli, F. and Izzo, A. A., “The Plant Kingdom As a Source of Anti-Ulcer Remedies,” Phytotherapy Research 14: 581-91 (2000).

For some patients exhibiting indigestion-type symptoms, no ulcer is found when they are subject to an internal examination. The term “non-ulcer dyspepsia” is often used for this condition. Although standard drugs like acid suppressants are sometimes beneficial for this condition, many dyspepsia patients do not gain any benefit, and herbal remedies have sometimes been tried. Licorice has been added to some of these herbal remedies. One commercially available herbal preparation (Iberogast, STW-5*) contains extracts from bitter candy tuft, chamomile flower, peppermint leaves, caraway fruit, licorice root, lemon balm leaves, angelica root, celandine herbs, and milk thistle fruit, and was found to help patients with functional dyspepsia in a double-blind placebo-controlled comparative study. Madisch, A., Melderis, H., Mayr, G. et al., “A Plant Extract and Its Modified Preparation in Functional Dyspepsia,” Z. Gastroenterology 39: 511-17 (July 2001).

Although sodium bicarbonate is not usually considered to be a “natural bioactive product,” the use of this very simple molecule may provide benefits if used in conjunction with a bioactive compound. For example, sodium bicarbonate has been shown to reduce the amount of gastric injury in rats and humans caused by aspirin if co-administered with the aspirin. Aspirin-bicarbonate solutions produced much less damage in starved rats than aspirin suspensions given at low (50 mg/kg body weight) or high therapeutic doses (200 mg/kg body weight). Rainsford, K. D., “Electronmicroscopic Observations on the Effects of Orally administered Aspirin and Aspirin-Bicarbonate Mixtures on the Development of Gastric Mucosal Damage in the Rat,” Gut 16: 514-27 (July 1975). It has also been found that villous damage of the proximal duodenum caused be histamines in rats was significantly inhibited by pretreatment with sodium bicarbonate given orally or cimetidine, omeprazole and NC-1300 given subcutaneously. Tanaka, H., Takeuchi, K. and Okabe, S., “Histamine-Induced Villous Damage in the Rat Duodenum, Japanese J. Pharmacol. 51(2): 291-97 (October 1989).

No established track record exists, however, for suggesting that soy products, licorice, or sodium bicarbonate, by themselves, are efficacious for treating inflammation or ulceration of the gastrointestinal system. One study has suggested that the addition of soybean trypsin inhibitor or casein may be beneficial by preserving the activity of artificially administered growth factors from digestion as they pass through the small intestines. See Playford, R. J., Woodman, A. C., Clark, P. et al., “Effect of Luminal Growth Factor Preservation On Intestinal Growth,” Lancet 341 (8849) ______ (1993). In a similar manner, scientists in another research study added pure soybean trypsin inhibitor to bovine colostrum in an effort to increase the passive transfer of antibodies from the milk into the calfs bloodstream by preventing the digestive enzymes present in the calf's intestines from digesting these suckled antibodies before they could be absorbed by the calf. Quigley, J. D. 3rd, Martin, K. R., Dowlen, H. H., and Lamar, K. C., “Addition of Soybean Trypsin Inhibitor to Bovine Colostrum: Effects on Serum Immunoglobulin Concentrations in Jersey Calves,” Journal Dairy Science 78: 886-92 (April 1995). However, any protective function provided by soybean trypsin inhibitor could not affect bioactive compounds applied to sites in the gastrointestinal system upstream of the small intestine, or if applied to sites outside of the gastrointestinal tract, such as skin or the cornea.

It is possible that many of the bioactive agents discussed above with respect to the gastrointestinal tract may also be applicable to the treatment of a variety of skin ailments, particularly those relating to virus-induced (including Herpes simplex and Varicella zoster viruses) and burn-induced problems. For example, cold sores are recurrent herpes simplex infection around the mouth. These lesions have a variable recurrence rate and may be associated with respiratory infections. Their occurrence may be also related to non-specific factors such as emotional stress, menstruation, and sunshine. For most patients who do not have an underlying immune defect (like leukemia or AIDS), the condition slowly resolves itself. For those individuals who get regular recurrences, drugs, which specifically inhibit the DNA replication of the virus (usually acyclovir or gancyclovir) may be taken by mouth or applied topically. This must be started at the earliest sign of an attack to be most effective.

There is evidence that prophylactic oral acyclovir (600-1000 mg per day in two doses) does seem to reduce the frequency and severity of cold sore attacks. An increase in the mean time to recurrence has been shown to occur, increasing from 46 to 1 18 days. However, the cost of oral acyclovir and its low efficacy in recurrent herpes labalis make it inappropriate for most non-immunocompromised patients in the community (British Medical Journal editorial, 1996). Prophylaxis should only be considered if attacks are sufficiently frequent, severe, prolonged, and psychologically distressing.

Chickenpox is a highly infectious, acutely contagious disease predominantly affecting children, though it may occur at any age. It is characterized by fever and a rash, and is caused by Varicella zoster virus. The name chickenpox is said to relate to the similarity of the skin lesions to boiled chickpeas.

The incubation period for chicken pox is from 14 up to 21 days, and is infectious from 4 days before the appearance of the rash until all lesions have scabbed over (approximately one week). It is very infectious. When the rash occurs, it may be macular (flat), popular (raised), or vesicular (small blisters) depending on age. Over the next few days and weeks, the vesicles dry and crust over, and sometimes scar if scratched to excess. More rarely, a heomorrhagic rash may occur in immuno-suppressed patients.

In the home situation, the usual treatment is to bathe the lesions with calamine lotion as an antipruritic (anti-scratch). The lesions can be bathed with antiseptic solutions like chlorhexidine. Oral acyclovir may also be given to immuno-competent adults and older adolescents with chickenpox. Acyclovir is not generally indicated for immuno-competent children in whom the disease is usually milder. Immuno-suppressed patients should be given immunoglobulin to Varicella zoster and acyclovir within two days of contact with varicella. If they develop chicken pox, they should be treated with acyclovir. Antibiotics should be given for secondary infections.

Shingles is an acute, unilateral (one sided), self-limiting inflammatory disease of cerebral ganglia and the ganglia of posterior nerve roots and peripheral nerves in a segmented distribution, caused by Varicella Zoster virus—the chicken pox virus. It is characterized by small vesicles in the cutaneous areas along the course of affected nerves. This can be thought of as a reactivation of varicella that has remained latent (dormant) in the sensory dorsal root ganglion.

Twenty percent of all adults suffer an attack of shingles at some time in their lives, and up to 2% may have a second episode. The incidence of this Varicella zoster reactivation problem increases with age: in early adult life there is a prevalence of 2-3 per 1,000, rising to 10.1 per 1,000 for people aged over 80.

The reason why an episode of shingles begins is often unclear although there are known predisposing factors such as immunosuppression caused by leukemia, HIV infection, or as a side effect of medical treatment (iatrogenic). Shingles cannot be caught directly from a patient with shingles although, somewhat counter-intuitively, in some susceptible individuals there is a risk of developing shingles following contact with a patient with chickenpox (the mechanism is unclear). The key clinical feature of shingles is a skin eruption of vesicles (small blisters) often preceded by pain and paraesthesia (tingling) by several days. Erythema (redness) precedes the development of vesicles. The vesicles may become pustular 2-3 days following eruption, and may separate a further 3 weeks afterwards. A tender lymphadenopathy (‘swollen glands’) is common in the early stages of the rash. There is increased itching and burning. The affected area may remain depigrnented and often it is hypoalgesic (reduced sensation). A widespread (“disseminated”) pattern similar to that of chickenpox, may also occur.

Shingles most commonly affects the lower thoracic region or the ophthalmic division of the trigeminal nerve (i.e., around the eye). Occasionally, motor nerves as well as sensory nerves are affected causing paralysis—for example facial paralysis in Ramsay Hunt Syndrome, or urinary retention.

Mild attacks of shingles usually require symptomatic treatment only. When medication is used, it usually comprises oral administration of acyclovir, although fancyclovir and valaciclovir are alternatives. A study comparing acyclovir 800 mg five times per day for 7 days against a control “placebo” tablet demonstrated that it reduced the duration of the rash and the acute pain in immuncompetent patients treated within 48 hours of the onset of the last vesicle. To be effective acyclovir must be given within 72 hours (at most) of the onset of the rash, until 48 hours after the appearance of the last lesion.

Acyclovir remains the treatment of choice for immunosuppressed patients or patients with ocular Herpes zoster, as there is no evidence relating to the effectiveness of the newer agents in these situations.

In addition to considering the possibility of applying natural bioactive compounds to viral-related skin injury, another potential application is for the prevention or treatment of burns. Burns to skin are very common. Such burns may arise from thermal, electrical, chemical, or inhalation-induced injuries. In the U.S., more than 2 million thermal injuries occur annually, requiring 70,000 hospital admissions, and causing more than 5,000 deaths. In the Third World, the rate of burn injury, morbidity, and mortality is far greater due to a variety of reasons, including more dangerous cooking and heating practices in such countries.

Minor burns are generally self-healing. Standard treatment for such burns aims to prevent dehydration and infection. Therefore, after cleaning the burned area of the skin with saline or an antiseptic, a silver sulphadiazine cream (Flamazine®) will be applied with the wound then covered by a thickly absorbent dressing. Burns which have not re-epithelialized within 14-21 days should be considered for skin grafting.

Being able to enhance the bioactive effect of colostrum and other bioactive compounds through the introduction of additive components for the treatment of inflammation, ulcers, and other cell injury disorders or ailments of the gastrointestinal system or skin would be very advantageous. Moreover, ensuring that such additives to the natural bioactive agents are, in turn, natural products themselves would be of great benefit to avoiding side effects in the treatment of such disorders or ailments to repair cell injuries.

### SUMMARY OF THE INVENTION

A method for prophylactically treating a gastrointestinal disorder or skin ailment in a mammal through the administration of an enhanced bioactive agent composition is provided according to the invention. For a gastrointestinal disorder, the enhanced bioactive agent composition comprises a bioactive agent in combination with at least one bioactivity-enhancing additive selected from the group consisting of a soy product, licorice product, and sodium bicarbonate. For a skin ailment, the enhanced bioactive agent composition comprises a bioactive agent in combination with a soybean product, although a licorice product may be added to assist in the penetration of the enhanced bioactive agent composition to the site of a virus-based skin ailment or the transfer of the enhanced bioactive agent composition to a cut, wound, abrasion, or burn in the skin. The bioactive agent preferably is bovine colostrum. The effective amount of enhanced bioactive agent composition to be used will depend upon such factors as the age and weight of the mammal, the bioactivity level of the bioactive agent, the gastrointestinal disorder or skin ailment at issue, and whether treatment of existing symptoms of the gastrointestinal disorder or skin ailment, or prevention of the onset of such symptoms is desired.

A medicament comprising an enhanced bioactive agent composition for prophylactically treating a gastrointestinal disorder or skin ailment in a mammal is also provided according to the invention.

### BRIEF DESCRIPTION OF THE DRAWINGS

In the accompanying drawings:

FIG. 1 is a series of schematic drawings showing normal and damaged epithelial, endothelial, or connective tissue, along with representations of the natural restitution, proliferation, and remodeling processes for healing such damaged tissue.

FIG. 2 is a graph comparing the effect of the same colostrum sample at different concentrations on cell proliferation, compared with a negative baseline control.

FIG. 3 is a graph comparing the effect of the same colostrum sample at different concentrations and the same soy protein sample at different concentrations on cell proliferation, compared with a negative baseline control.

FIG. 4 is a graph comparing the effect of samples of colostrum (1 mg/ml), soy protein (0.1 mg/ml), and licorice (0.1 mg/ml) on cell proliferation, compared with a negative baseline control.

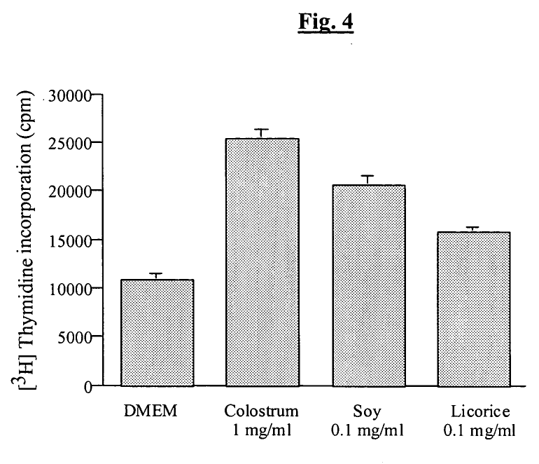

FIG. 5 is photographs showing the effect of colostrum on cell restitution, compared with a negative baseline control.

FIG. 6 is a graph comparing the effect of the same colostrum sample at different concentrations on cell restitution, compared with a negative baseline control, over time.

FIG. 7 is a graph comparing the effect of samples of colostrum (1 mg/ml), milk powder (1 mg/ml), and EGF (10 μg/ml) on cell restitution, compared with a negative baseline control.

FIG. 8 is a graph comparing the effect of samples of licorice (1 and 2 mg/ml) and EGF (10 μg/ml) on cell restitution, compared with a negative baseline control.

FIG. 9 is a graph comparing the effect of various samples of colostrum, soy, EGF, and combinations of colostrum and soy on cell restitution, compared with a negative baseline control.

FIG. 10 is a graph comparing the effect in a rat study of colostrum, soy, and a combination of colostrum and soy on gastric damage, compared with a saline control.

FIG. 11 is a graph comparing the effect in a rat study of sodium bicarbonate on gastric damage, compared with a saline control.

### DETAILED DESCRIPTION OF THE PREFERRED EMBODIMENT

Use of compositions comprising a bioactive agent in combination with a soybean, licorice, and/or sodium bicarbonate product for the prophylactic treatment of a disorder of the gastrointestinal system or skin ailment is provided by the invention. Such invention may take the form of a method for administering an effective amount of such an enhanced bioactive agent composition to a patient (be it animal or human), who is suffering from a gastrointestinal disorder or skin ailment to prophylactically treat the symptoms and tissue injury related to such disorder. Likewise, the invention may take the form of a specific enhanced bioactive agent composition that is efficacious for the prophylactic treatment of a gastrointestinal disorder or skin ailment.

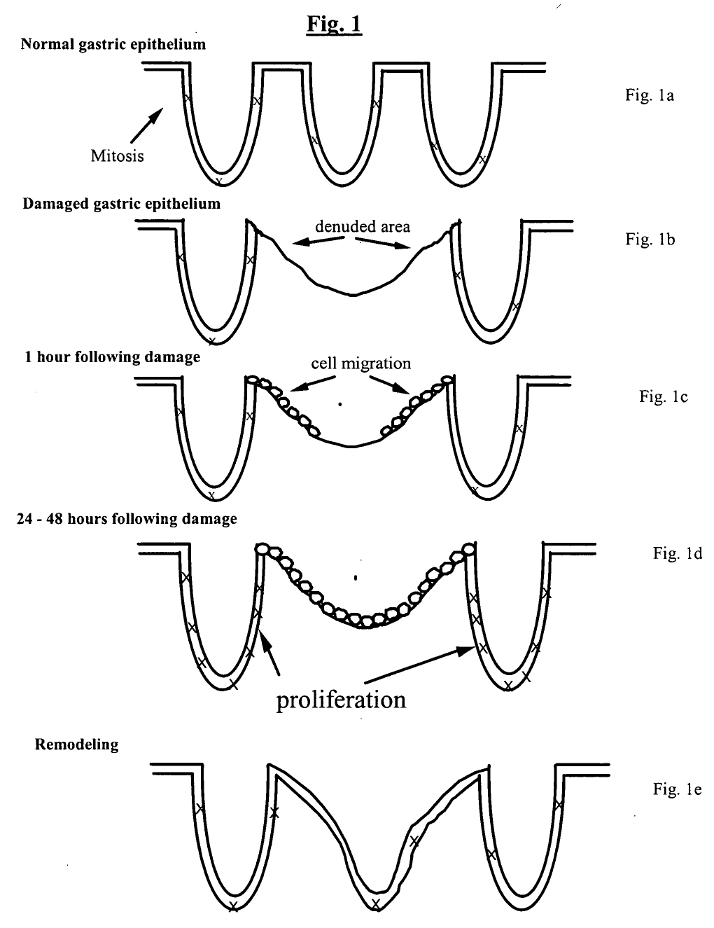

FIG. 1 shows the three phases of the healing process when an injury occurs to epithelial, endothelial, or connective tissue cells. For example, the normal gastric epithelium is represented in FIG. 1a. On the other hand, an injury is represented by the denuded region of the gastric epithelium shown in FIG. 1b. Within 1 hour after the injury, a rapid response will typically occur, involving migration of surviving cells from the wound edge to cover the denuded area, as shown in FIG. 1c. This step is termed “restitution.” One to two days after the injury has occurred, a much slower increase in the number and differentiation of cells takes place to finish filling the damaged region of the gastric epithelium (FIG. 1d). This second stage is called “proliferation.” The final stage of the healing process is “remodeling” (FIG. 1e) in which the mucosa slowly reestablishes an essentially normal looking mucosa along the surface of the gastric epithelium.

For purposes of the present invention, “functional cells” means any cells relevant to the defense, normal structure, or repair of mammalian animals, including epithelial, endothelial, connective tissue, and inflammatory cells. HT-29 and keratinocytes are examples of epithelial cells. Mouse pancreatic islet endothelial cells (MS 1) and human endothelial cells derived from the umbilical vein are examples of endothelial cells. Connective tissue cells include fibroblasts and vascular smooth muscle cells.

In the context of the present invention, a “gastrointestinal disorder” means any medical condition caused by injury to the functional cells of a site in the gastrointestinal system, including but not limited to: apthous ulcers on the surface of the mouth; esophagitis (acid reflux) of the esophagus; gastritis or gastric ulcers in the stomach, whether caused by bacteria (e.g. Helicobacter Pylori) or chemicals (e.g., NSAIDs or iron supplements); duodenal ulcers, duodenitis, Chrohn's Disease (a form of inflammatory bowel disease, IBD) or Celiac Disease (allergy to wheat) in the small intestine; ulcerative colitis (a form of IIBD usually restricted to the large intestine), microscopic colitis, collagenous colitis, or diverticular disease in the large intestine; or non-ulcer dyspepsia in the stomach or small or large intestine. In addition, various gastrointestinal disorders can affect both small and large intestine and include conditions such as “increased gut permeability syndrome,” also known as “leaky gut syndrome,” graft versus host disease or other autoimmune disease such as rheumatoid arthritis that affect the gut. The common denominator of all of these gastrointestinal disorders is injury to cells, whether by inflammation, ulceration, or otherwise, that must be treated by means of cell restitution or cell proliferation as part of the repair process.

For purposes of the present invention, “skin ailment” means any medical condition caused by injury to the functional cells of a site on the skin of a mammal, including but not limited to: viral-based cold sores, chickenpox, or shingles; thermal, electrical, or chemical-induced burns to the exterior surface of the skin; or cuts, wounds, or abrasions to the skin.

For purposes of the present invention, “bioactive agent” means any natural or manufactured substance that is effective for enhancing the restitution and/or proliferation of cells that has been damaged. The cause of such damage may include, but is not limited to, gastric ulcers, peptic ulcers, IBD, necrotizing enterocolitis, short bowel syndrome, apthous ulcers, cold sores, chicken pox, shingles, and skin burns. Such bioactive agents may constitute natural unicellular or multicellular products plant, animal, marine, or insect origin, or derivatives from such natural products. The recently published article Gosh, S. and Playford, R. J., “Bioactive Natural Compounds for the Treatment of Gastrointestinal Disorders” Clinical Science 104: 547-56 (2003) discusses a number of these compounds in some detail, and is hereby incorporated by reference. Examples of such bioactive agents include:
Bacteria and Yeasts: Probiotics; botulinum toxin from the anaerobic baceteria Clostridium botulinum.
Plant Sources: Prebiotics like chicory root, non-digestible oligosaccharides, and low-digestable carbohydrates; symbiotics entailing products in which a prebiotic and a probiotic are combined; aloe vera and some of its individual constituents such as acemannan; soybean and its derivatives; tumeric and its individual constituents such as Curcumin; bael (Aegle marmelos); garlic, pine bark extract; dragon's blood (Sangre de grado).
Other plant derivatives include dithiolthiones, glucosinolates and isothiocyanates (cruciferous vegetables), coumarines, and limonene (citrus fruits), isoflavones inositol hexaphosphate, protease inhibitors and saponins (soybean), carotenoids (palm oil, yellow vegetables) and allium compounds (onion, garlic and leek) and prebiotics such as inulin, fructo-oligosaccharides and soybean oligosaccharides and Chicory fructo-oligosaccharides (ChiFos).
Animal Sources: Colostrum and derivatives obtained from it or artificially produced such as cytokines, including interleukin (IL)-1beta, IL-6, IL-10, TNF-alpha, and granulocyte macrophage colony stimulating factors, nucleosides and nucleotides, and a variety of other growth factors including transforming growth factor alpha and beta, insulin like growth factor 1 and 2, epidermal growth factor, and granulocyte colony stimulating factor.
Milk derived products such as caseins and their subfractions (αₛ₁, αₛ₂, β and κ caseins), whey and its subfractions α-lactalbumin, β-lactoglobulin, lactoferrin, lactoperoxidase, immunoglobulins, glycomacropeptide and a variety of growth factors, including the EGF-receptor ligand, beta-cellulin.
Blood serum obtained from mammals like cows, pigs, goats, or rabbits.
Other animal sources such as Deer antler and velvet.
Marine Sources: Fish oils such as eicosapentaenoic and docosahexanenoic acids; proteins, amino acids and other subcomponents obtained or derived from fish origin; derivatives obtained from other marine sources (such as Sponges and snails) including manoalide, and contignasterol.
Insect Sources: Honey; royal jelly.
Other Sources: Proteins, peptides and other factors produced naturally or artificially produced from bacterial, yeast, mammalian or insect cells based on natural products such as epidermal growth factor, trefoil peptides, and pancreatic secretory trypsin inhibitor. Derivatives of such products such as modifying amino acid sequence to improve stability or activity or complexing with products such as zinc, iron, etc.

For each of these potential sources, application may also apply to derivatives such as heat-treated sources that have undergone partial extraction using methods like alcohol extraction, and sources that have undergone partial or complete enzymatic or mechanical digestion.

As used in this application, an “enhanced bioactive agent composition” means any preparation for the prophylactic treatment of a gastrointestinal disorder or skin ailment comprising at least one bioactive agent in combination with at least one of the following: a soybean product, a licorice product, and/or sodium bicarbonate.

For purposes of the present invention, a “soybean product” means soy flour or any other soy product like soy protein isolate, soy protein concentrate, isolated soy protein, soy milk, hydrolyzed soy proteins, soybean oil, lecithin, defatted soy flakes, tofu, or a subcomponent of soy, such as trypsin inhibitors, genistein, isoflavones, or lectins derived therefrom, whether administered in its native form or in a modified form, such as heat-treated, hydrolyzed, or partially extracted. Soy flour, soy milk, hydrolyzed soy proteins, defatted soyflakes, and tofu can be added in the range of about 2-25% by weight to the end product containing the enhanced bioactive agent composition. Likewise, soy protein isolate, soy protein concentrate, and isolated soy protein can be added in the range of about 2-20% by weight to the end product containing the enhanced bioactive agent composition. Finally, trypsin inhibitor, genistien, isoflavoness, lectins, and other soybean subomponents should be added in the range of about 0.005-5.0% by weight to the end product comprising the enhanced bioactive agent composition. One value of adding the trypsin inhibitor is that it will help to protect growth factors and other peptides contained in the enhanced bioactive agent compositions from digestion so that they can be delivered to the affected areas of the gastrointestinal system.

For the purposes of this invention, a “licorice product” means natural licorice root extract, DGL, and any hydrolized or ester amide derivatives thereof. Any such licorice product should be added in the range of 0.5-25% by weight to the end product comprising the enhanced bioactive agent composition. Licorice is known to assist in skin penetration by drugs, so it may be useful for enhanced bioactive agent compositions applied to skin ailments like cold sores and skin wounds. Licorice may also help with some of the non-ulcer dyspepsia symptoms associated with peptic ulcers and gastric ulcers.

For purposes of this invention, sodium bicarbonate should be added in the range of 0.5-25% by weight to the end product containing the enhanced bioactive agent composition. Sodium bicarbonate has the advantages of helping to neutralize some of the acids prevalent in the stomach, and may also enhance the palatability of an enhanced bioactive agent composition due to its “fizzy” action. The sodium bicarbonate may also act to protect the growth factors contained in the bioactive agent from partial or complete degradation due to the harsh environment of the stomach. Its major value is therefore likely to be in its use to enhance activity for gastrointestinal disorders, especially those of the upper gastrointestinal tract, rather than in its use for skin ailments or via enema therapy.

An “effective amount of an enhanced bioactive agent is an amount sufficient to prevent, treat, reduce, and/or ameliorate the symptoms and/or underlying causes of a gastrointestinal disorder or skin ailment. In some instances, an “effective amount” is sufficient to eliminate the symptoms of a gastrointestinal disorder or skin ailment and, perhaps, overcome the gastrointestinal disorder or skin ailment, itself. In the context of the present invention, the terms “treat” and “therapy” and the like refer to alleviate, slow the progression, prophylaxis, attenuation, or cure of existing gastrointestinal disorder or skin ailment symptoms. “Prevent,” as used herein, refers to putting off, delaying, slowing, inhibiting, or otherwise stopping, reducing, or ameliorating the onset of such gastrointestinal disorder or skin ailment symptoms. “Prophylactic treatment,” as used herein, means either the administration of the remedy in the absence of gastrointestinal disorder or skin ailment symptoms to prevent the onset or occurrence of gastrointestinal disorder symptoms within the mouth, esophagus, stomach, large or small intestine, or skin, or the treatment of gastrointestinal disorder symptoms that already exist within the mouth, esophagus, stomach, large or small intestine or skin, using the remedy.

For purposes of this invention, the mammal to whom the enhanced bioactive agent composition is to be administered is preferably a human, but may also include domesticated animals like dogs, cats, and horses, and livestock like pigs and cows.

Colostrum shall be used as an exemplary bioactive agent for purposes of this application and the enhanced bioactive agent composition claimed hereunder, but it is important to appreciate that any bioactive agent product that influences cell migration or proliferation can be applied to the enhanced bioactive agent composition. Colostrum is the first milk produced after birth, and is particularly rich in non-specific and specific anti-microbial factors like immunoglobulins and other bioactive molecules including non-peptide factors, such as nucleotides, and a whole variety of peptide growth factors such as epidermal growth factor (“EGF”), transforming growth factor-alpha (“TGF-α”), insulin-like growth factors I and II, vascular endothelial growth factor, platelet-derived growth factor, and lactoferrin. In combination with the milk that is subsequently produced by the mother, colostrum is an important contributor to the nutrition, growth, development, and immunological defense of the newborn infant.

While colostrum may be sourced from any mammal for purposes of this invention, such as cows, goats, sheep, and buffalo, it preferable is sourced from a cow. The immune and growth factors found in such bovine colostrum are virtually identical to those found in human colostrum, and the immune factors are reportedly four times richer. An additional benefit to bovine colostrum is its special glyco-proteins and protein constituents, which have been shown to be extremely effective in protecting colostrum's active components from the destructive forces of the human body's digestive system.

Bovine colostrum is commercially available from a number of sources. Such manufacturers include Sterling Technology, Inc. of Brookings, S.D.; Immuno-Dynamics of Iowa, Inc. of Perry, Iowa; and Labelle Industries of Ripon, Calif.

The newborn calf requires three quarts of colostrum within the first six hours after birth, and then two quarts within the next twelve hours, to ensure its health and vitality. Therefore, less commercial colostrum for human consumption will be obtainable during this first six-hour time period from the mother cow. After 72 hours, the colostrum starts to become much more like full milk with complete absence or much reduced amounts of the beneficial factors that are important to human health maintenance and restoration. Therefore, the bovine colostrum for purposes of this invention should preferably be collected from the cow within the first 72 hours after birth of the calf, more preferably within the first 48 hours after birth, even more preferably within the first 24 hours after birth, most preferably within the first two milkings following parturition.

It may be beneficial to use colostrum that was obtained from cows that have been certified as free from hormones, pesticides, and antibiotics, so as not to increase other health problems in a person who consumes the colostrum. There are a number of certified, organic dairies in the United States, New Zealand, and other countries that can produce organic colostrum that fits this criterium.

The colostrum of the present invention should also be minimally processed at the lowest temperature possible in order to avoid destruction of its natural components or denaturation of the product. For example, colostrum in liquid form will typically be manufactured as follows: colostrum from the cow will be frozen by the farmer and sent to the colostrum manufacturer. The manufacturer will then thaw the colostrum in order to grade, test, and analyze it for bioactivity and other desired specifications; heat treat the colostrum at approximately 162° F. for 16 seconds; subject the heat-treated colostrum to mechanical separation and removal of the fat component; enzymatic or acid treatment to remove casein; and filtration to yield the final liquid colostrum product. Because such liquid colostrum will be less stable than powdered colostrum, and more subject to spoilage, it will need to be kept at a temperature around 4 degrees centigrade (refrigerator temperature) in order to ensure that it does not spoil.

Alternatively, the colostrum may be spray dried to form a powder, as is known within the industry. U.S. Pat. Nos. 3,956,521 and 4,281,024, both owned by Aktieselskabet Niro Atomizer, describe processes and equipment for spray drying milk products to produce powders, and are incorporated herein by reference. In order to produce such powdered colostrum, colostrum from the cow will typically be processed as described above through the heat-treatment step. It will then be cooled down and then spray dried. The resulting colostrum powder will be a stable product that can be stored under normal ambient conditions without spoiling or degradation of the vital components in the colostrum, although it may also be defatted to provide additional storage stability. A principal advantage of powderized colostrum is that it may be conveniently contained in a capsule or tablet using technology that is readily known in the art. Alternatively, the colostrum may be purchased as a bulk powder that can be added in a measured amount to water, a drink, or food item for easy consumption. Examples of such drinks include milkshakes, protein shakes, drink concentrates, and fruit juices. Examples of such food items include energy bars, candy bars, yoghurt, kefir, cottage cheese, soft cheese, and ice cream. Liquid colostrum may be drunk by itself in a measured amount by the patient, or added in a measured amount to another drink or food item, such as the ones listed above.

Finally, it is envisioned under this invention that the colostral composition may be prepackaged in a measured amount in a drink or food item, so as to administer for convenience the correct dosage of the colostral composition to a patient for prophylactically treating IBS. The possible list of prepackaged drink or food items includes, but is not limited to, those mentioned above. Co-pending application U.S. Ser. No. ______ filed on Jul. 16, 2004 by the inventor of the present application discusses in greater detail preparation, storage, and manufacturing methods for liquid, powder, tablet, and capsule forms of colostrum products, and its specification is incorporated by reference into the present application.

One or more other additives may advantageously be added to the enhanced bioactive agent composition of the present invention to improve or enhance its characteristics or performance. For example, because colostrum can have a natural off flavor, a natural or artificial form of a flavoring agent like licorice, cherry, orange, chocolate, strawberry, vanilla, butter, or lemon can be added to the liquid or powdered colostrum product to make it more palatable to swallow. Approximately 1 ml of the flavoring agent per 5 ounces of liquid colostral composition or 200 g of colostral composition powder could be used.

Thickening agents, particularly hydroxymethyl cellulose, methylcellulose, or guar gum, may be added to the enhanced bioactive agent composition to enhance the retention time of the enhanced bioactive agent composition in the stomach or if applied in an enema formulation, or if applied topically to the skin.

Colostrum can also be straw-colored, which can look unappealing. Thus, a coloring agent can be added to the liquid or powdered colostrum product in order to encourage a person to consume it. A number of natural and artificial colorants are well known within the food industry, and therefore available to the practitioner of this invention. Coloring agents may also be beneficial to gelatin capsules containing colostrum.

For purposes of the present invention, the enhanced bioactive agent composition may also be “spiked” with a pure growth factor or immune modulator, such as EGF, transforming growth factor-α (“TGF-α”), transforming growth factor-β (“TGF-β”), pancreatic secretory trypsin inhibitor, glucagon-like peptide 2 (“GLP2”), or its analogues, growth hormone, GMCSF, hSP, interleukin-10, or lactoferrin. The soybean product will help to protect these additives from digestion in the stomach and the small intestine. Colostrum contains multiple factors that may work together to assist in healing cellular injuries to the gastrointestinal system or the skin. For example, colostrum contains EGF and lactoferrin that have been shown to stimulate cell restitution more quickly than if EGF or lactoferrin were used alone. Use of colostrum should therefore facilitate faster healing than use a single peptide. Therefore, spiking a bioactive agent like colostrum with an additional amount of a peptide like EGF or lactoferrin should further enhance the rate of cell restitution to prophylactically treat the gastrointestinal disorder or skin ailment.

Depending upon the indication intended to be prophylactically treated by the enhanced bioactive agent of the present invention, the composition of that enhanced bioactive agent composition will vary. For example, the active ingredients of an enhanced bioactive agent composition for an ulcer or inflammation-induced gastrointestinal disorder should include at least one bioactive agent in combination with at least one of the following additives: (1) a soy product; (2) a licorice product; or (3) sodium bicarbonate. For a virus-induced skin ailment, the active ingredients of the enhanced bioactive agent composition should include at least one bioactive agent in combination with at least one soy product. A licorice product may be further incorporated into the enhanced bioactive agent composition to aid in the penetration through the skin of the active ingredients. For skin wounds or burns, the active ingredients of the enhanced bioactive agent composition should include at least one bioactive agent in combination with at least one soy product. A licorice product may be further incorporated into the enhanced bioactive agent composition as a transfering agent.

For purposes of this invention, the bioactive agent portion of the enhanced bioactive agent composition must possess sufficient biological activity to increase cell restitution or proliferation in a relevant cell culture model by at least 10% above the baseline level of movement or growth for corresponding cells in a culture medium without the bioactive agent or any other pro stimulant. Not all enhanced bioactive agent composition products, or even samples of the same enhanced bioactive agent composition product, exhibit the same level of biological activity because bioactivity of an enhanced bioactive agent composition is affected by its preparation, storage, and manufacturing practices. Therefore, it is insufficient merely to focus upon total protein and immunoglobulin levels as is typically done within the colostrum industry. It is well known in the art to employ a “proliferation” assay using intestinal cells and thymidine incorporation to measure cell growth. Moreover, it is well known in the art that a “restitution” assay using intestinal cells where a standard wound has been induced followed by determination of how quickly the cell gap around the wound closes may be employed to measure cell movement. See Playford, R. J., Floyd, D. N., MacDonald, C. E., et al., “Bovine Colostrum Is a Health Food Supplement Which Prevents NSAID Induced Gut Damage,” Gut 44: 653-58 (1999). Co-pending application U.S. Ser. No. ______ filed on ______ 2004, by the inventor of the present invention provides a novel and reproducible scoring method for the bioactivity of bioactive agents like colostrum, and its specification is hereby incorporated by reference into the present application.

The soy product and/or licorice product should then be added to the bioactive agent constituent in the enhanced bioactive agent composition by a sufficient amount to improve the cell restitution and/or proliferation parameter by a further 10-200% above the baseline level, preferably 50-100% above such baseline level, most preferrably 60-80% above such baseline level.

Note, however, that for sodium bicarbonate addition, the restitution and proliferation cell culture models are less relevant than whole animal models for purposes of determining enhanced levels of bioactivity. Therefore, the value of sodium bicarbonate to enhance bioactivity can alternatively be tested by measuring its ability to reduce the amount of gastric injury by at least a further 10%, preferably 50-100%, more preferably 60-80% below that of using the bioactive agent, alone. Such methods are well known in the art, for example, using the indomethacin-induced gastric damage model reported in Playford, R. J., Floyd, D. N., MacDonald, C. E. et al., “Bovine Colostrum Is a Health Food Supplement Which Prevents NSAID-Induced Gut Damage,” Gut 44:653-58 (May 1999). An example of the use of this system is provided later in Example IX.

Alternatively, if the bioactive agent exhibits no cell restitution, proliferation, or gastric damaging preventative activity of its own, then the soy product or licorice product should be added to the enhanced bioactive agent composition by an amount sufficient to produce a further 10% increase above the level that pertains to using the soy product or licorice product on its own. Likewise, sodium bicarbonate should be added to the enhanced bioactive agent composition by an amount sufficient to produce a further 10% reduction below the level that pertains to using sodium bicarbonate on its own.

Provided that the enhanced bioactive agent composition exhibits such a minimal level of bioactivity, it may be administered to a patient as a prophylactic treatment for a gastrointestinal disorder or skin ailment in any of the forms discussed above. In the case of a human age 16 or above averaging 70 kg in weight, the enhanced bioactive agent composition in liquid form should be administered in a single or multiple daily doses amounting in total to 1-200 ml/day, more preferably 5-100 ml/day, even more preferably 10-100 ml/day. If applied topically as a cream for skin wounds, cold sores, chicken pox, shingles, etc., the enhanced bioactive agent composition should be administered at a rate of 0.2-20.0 ml-day, preferrably 0.5-10.0 ml/day. In the case where colostrum is used as the bioactive agent in powdered form, the single or multiple daily dosage of the enhanced bioactive agent composition should total 1-100 g/day, more preferably 3-20 g/day, even more preferably 10-15 g/day. Where other bioactive agents are used, the appropriate daily dosage will obviously vary. However, in general terms, most naturally-derived compounds demonstrating bioactivity have tended to do so on the order of mg/ml, as opposed to μg/ml or g/ml, when analyzed in the cell culture restitution and proliferation assays. It is therefore likely that the vast majority of powdered doses of bioactive agents for purposes of this invention should be covered by extending the range for colostrum by five-fold, more preferably 3-fold, in each direction. For a five-fold conversion, this translates to 0.2-500 g/day, more preferably 0.6-100 g/day, more preferably 2-75 g/day. For a three-fold conversion, this translates to 0.33-300 g/day, preferably 1-60 g/day, more preferably 3.33-45 g/day. It is believed that a person of ordinary skill in the art should be able to determine the appropriate dosage within this outside range for a specific bioactive agent without undue experimentation.

To gain more precision in making this determination, the enhanced bioactive agent composition can be analyzed using the restitution and/or proliferation assays described above. The concentration (mg/ml) for a specific bioactive agent required to be used in these assay systems to increase cell movement or proliferation by about 75% above the value seen in the negative baseline control wells can then be employed to calculate the appropriate total daily dosage for the enhanced bioactive agent composition as follows:
Total daily dose=C_{75%}×10,000
where C_{75%}=Concentration of the enhanced bioactive agent composition need to stimulate the cells by 75% above the baseline value in the restitution or proliferation assay.
In the case of colostrum, this value is about 1 mg/ml in the cell culture assay systems, therefore giving a daily dosage of 10 g for the enhanced bioactive agent composition containing the colostrum.

Table 1 below shows the preferred daily dosage of the enhanced bioactive agent composition, and amounts of sodium bicarbonate, soy product, licorice product, and thickening agent used therein, for skin ailments, gastrointestinal disorders, and enema applications. The absolute amount of dry weight total volume required varies with the enhanced bioactive agent composition, but can be determined from the information provided above. Note that the amounts specified in Table 1 are treatment doses for an adult 70 kg human. Preventive treatment should require 50% of these amounts, and children under age 16 should receive half adult doses. Conversion of these doses for animals should be based upon the weight of the animal, as is well known in the art.

The enhanced bioactive agent composition may also be effective for preventing the onset or reducing the incidence of gastrointestinal disorder or skin ailment when taken on a regular basis over time. This could be a daily, in one or divided doses or taken on an intermittent basis. For example, a human age 16 or above averaging 70 kg in weight who is not suffering yet from gastrointestinal disorder or skin ailment symptoms should take single or multiple doses amounting in total to 2-70 ml/day, more preferably 10-30 ml/day, even more preferably 10-15 ml/day, as a preventive treatment against the onset of gastrointestinal disorder symptoms. The corresponding total enhanced bioactive agent composition dosage on a powder basis would be 330 mg-33 g g/day, more preferably 1-7 g/day, even more preferably 2-4 g/day.

For children younger than age 16, the dosages discussed above for treating or preventing gastrointestinal disorder should be cut in half. The dosages may also be adjusted to take into account the weight of the patient, as is well known in the medical arts. This is particularly important for administering the colostral composition to patients that are not humans.

TABLE 1

| | | Total Daily | Total Daily | | | | |
|---|---|---|---|---|---|---|---|
| | Total Daily | Dosage of | Dosage of | | | | |
| | Dosage of | Enhanced | Enhanced | | | | Optional |
| | Enhanced | Bioactive | Bioactive | | | | Thickening |
| | Bioactive | Agent | Agent | | | | Agent |
| | Agent | Composition | Composition | Sodium | | Licorice | (e.g., |
| | Composition | for Treatment | for Prevention | Bicarbonate | Soy (% | (% Dry | HMC) (% |
| | (ml) | (g) | (g) | (% Dry Wt.) | Dry Wt.) | Wt.) | Dry Wt.) |
| Skin | 0.2-20; | 0.3-150; | 0.3-75; | 0.5-10; | 2-25; | 0.5-25; | 5-50; |
| Ailment | Pref. 0.5-10. | Pref. 1-30; | Pref. 1-15; | Pref. 0.5-2.0. | Pref. 5-20; | Pref. 1-5; | Pref. 5-25. |
| | | MPref. 1-20. | MPref. 1-10. | | MPref. | MPref. 1. | |
| | | | | | 10. | | |
| GI | 1-200; | 0.3-300; | 0.3-150; | 0.5-30; | 2-25; | 0.5-25; | 2-25; |
| Disorder | Pref. 5-100; | Pref. 1-60; | Pref. 1-30; | Pref. 0.5-20; | Pref. 5-20; | Pref. 0.5-5; | Pref. 2-10; |
| | MPref. 10-100. | MPref. 3-45. | MPref. 3-22 | MPref. 1-15. | MPref. | MPref. 1. | MPref. 5. |
| | | | | | 10. | | |
| Enema | 1-200; | 0.3-300; | 0.3-150; | 0.5-10; | 2-25; | 0-10; | 2-25; |
| | Pref. 5-100; | Pref. 1-60; | Pref. 1-30; | Pref. 0.5-2.0. | Pref. 5-20; | Pref. 0-5. | Pref. 2-10; |
| | MPref. 10-100. | MPref. 3-45. | MPref. 3-22. | | MPref. | | MPref. 5. |
| | | | | | 10. | | |

“Pref.” = “preferably.”
“MPref.” = “more preferably.”

The following examples further illustrate the enhanced bioactive agent composition of the present invention.

### EXAMPLE I

### Effect of Colostrum as a Bioactive Agent on Cell Proliferation in Intestinal Cells

As discussed above, an increase in the rate of cell division plays a key role in reestablishing a normal mucosa along the epithelial or endothelial tissue lining following an injury (FIG. 1d). Cell culture models have traditionally been used as surrogate markers for this proliferation response. Because thymidine is a natural constituent of the DNA culture, thymidine incorporation is commonly used as a marker of proliferation. Cells that are actively dividing will therefore increase their uptake of thymidine in the preparatory state of cell division.

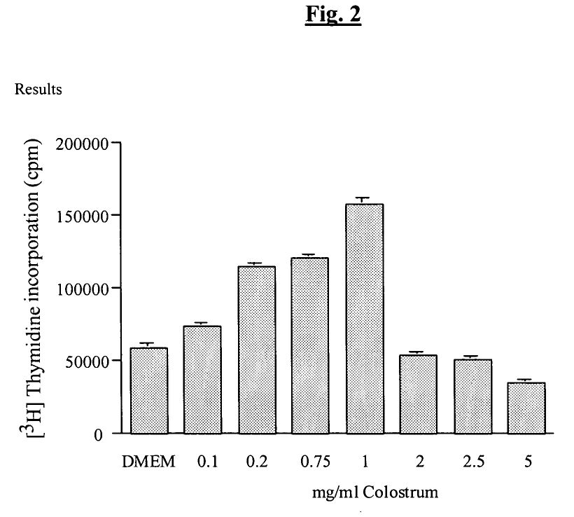

FIG. 2 demonstrates the results of a typical experiment. The human colonic cancer cell line HT-29 obtained from the European Collection of cell Cultures (www.ecacc.org) was grown in a solution of DMEM-containing glutamine and 10% fetal calf serum. The effects of a commercially available colostral preparation obtained from Sterling Technology of Brookings, S.D. tested at seven different concentrations (0.1, 0.2, 0.75, 1.0, 2.0, 2.5, and 5.0 mg protein/ml) were subsequently tested under serum-starved conditions. In order to assess the percentage of cells entering DNA synthesis, [³H]-thymidine (2 μCi/well) was introduced 24 hours after the addition of nothing (negative control) or the seven different colostrum preparations, and the cells were left for a further 24-hour period. For each condition, the stimulatory or inhibitory effect of the solutions was measured in six separate wells. Cell viability, determined by the ability to exclude 0.2% trypan blue, was greater than 90%.

The results are shown in FIG. 2. In wells in which no colostrum was added (i.e., just DMEM culture medium alone—the negative “baseline” control), thymidine incorporation was about 60,000 counts per minute (“CPM”) (column 1). Columns 2-8 show the effect of adding different concentrations of colostrum to the wells. As is often seen for agents with growth factor activity, the cell proliferation assays gave a typical “bell-shaped” dose response curves. As can also be seen, doses of colostrum added at a final concentration of 0.2, 0.75, and 1.0 mg protein/ml gave a markedly enhanced proliferation rate, as shown by the uptake in thymidine incorporation rising to about 170,000 CPM (columns 3, 4, and 5).

### EXAMPLE II

### Effect of Soy Protein on Cell Proliferation of Intestinal Cells

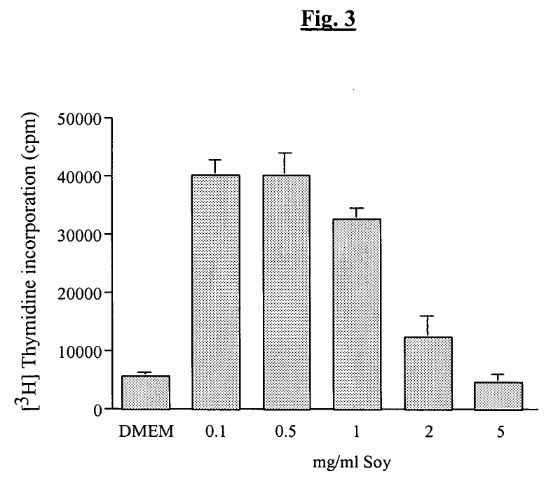

HT-29 cells were grown in the same manner as for the Example I protocol, except that different amounts of soy protein isolate (Cargill's PROLISSE ISP-521 IDP) were added to the wells instead of colostrum, at concentrations ranging from 0.1 to 5 mg protein/ml. The results for thymidine incorporation for these soy protein samples and the negative control are shown in FIG. 3. As can be clearly seen, a typical bell-shaped dose response curve is present with maximal stimulatory effect being found in the range of 0.1-1.0 mg protein/ml.

### EXAMPLE III

### Effect of Colostrum, Soy Protein, and Licorice on Cell Proliferation in Intestinal Cells

To examine the effect of licorice on cell proliferation and begin to examine its relative efficacy against colostrum and soya, the following experiment was performed. HT-29 cells were grown in the same manner as for Examples I and II, except that a single dose of colostrum (1 mg protein/ml), soya (0.1 mg protein/ml) and licorice (0.1 mg protein/ml) were added to different wells. The doses of 1 mg protein/ml colostrum and 0.1 mg protein/ml soya were chosen based on the results of the preliminary studies shown as Examples I and II. As can be clearly seen, the colostrum, soy and licorice all increased the amount of thymidine incorporation compared to the negative control (DMEM).

### EXAMPLE IV

### Effect of Colostrum as a Bioactive Agent on Cell Migration of Intestinal Cells

As discussed above, one of the earliest biological repair responses following injury to tissue cells is the migration of surviving cells over the denuded area caused by the injury to reestablish epithelial integrity (See FIG. 1c). Since it is extremely difficult to study this effect upon organ tissue inside a human or animal, cell culture models are commonly used as surrogate markers of this pro-migratory response. Several cell lines are potentially available for performing these studies, which include human colon cells such as HT-29 and caco2 cells, human intestinal lines like HIE cells, rat intestinal cell lines like RIE6, gastric cell lines like AGS and other similar cell lines such as IEC-6 and IEC 17 cells, T84 and NRK cells. The generality of these results allow them to be applied to the study of cell migration responses for human applications of bioactive agents like colostrum, and for the testing of such products for animal applications.

Accordingly, the human colonic cancer cell line HT-29 was grown to confluence in six well plates in a solution of DMEM-containing glutamine and 10% fetal calf serum. The monolayers of the cells were then wounded by scraping a disposable pipette tip across the dishes, washed with fresh serum-free medium, and then cultured in a serum-free medium in the presence of 1 mg/ml, 2 mg/ml, and 5 mg/ml colostrum solutions. The rate of movement of the anterior edges of the wounded monolayer cells was then determined by taking serial photomicrographs at various times after wounding (i.e., 0, 4, 8, 12, and 24) hours, using an inverted Nikon TS 100 microscope and a Nikon Coolpix 800 digital camera with 100-fold magnification. Identical regions were examined at each time point by premarking the base of the plates to facilitate alignment. Twenty measurements per field were performed by placing a transparent grid over the photomicrograph, and measuring the distance moved by the anterior edge of the cells from the original wound line. Each wound was examined in at least three different regions, and expressed as mean and SEM of three separate experiments.

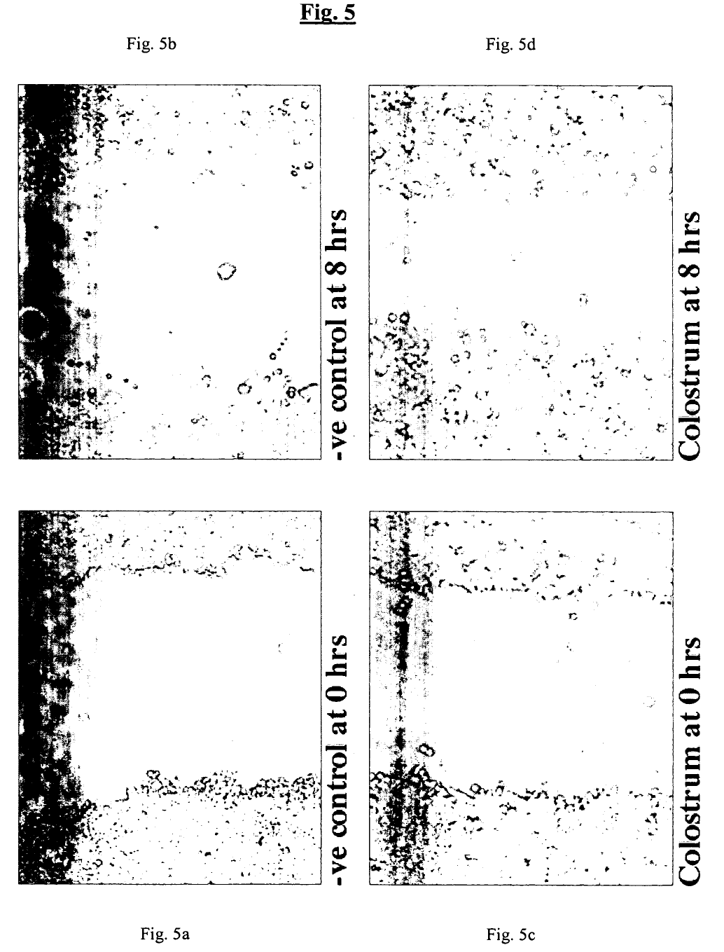

FIG. 5 shows pictures taken at the start of an experiment (FIGS. 5a and 5c) and eight hours later (FIGS. 5b and 5d) of cells that were grown in culture medium containing DMEM alone (FIGS. 5a and 5b) versus those where commercial colostrum sourced from Sterling Technology, Inc. of Brookings, S.D. was added to the cell culture media at 1 mg protein/ml (FIGS. 5c and 5d). As can be clearly seen, the gap produced by wounding in the negative control (DMEM alone) well started off as roughly the same size as the gap in the well of cells to which the colostrum was added. However, eight hours later, the gap was much smaller in the wells where the colostrum was added, reflecting an increase in the rate of movement of the anterior edge of the cell monolayer, compared with the negative control well. Indeed, by 24 hours, the gap completely closed in wells where colostrum was added (not shown).

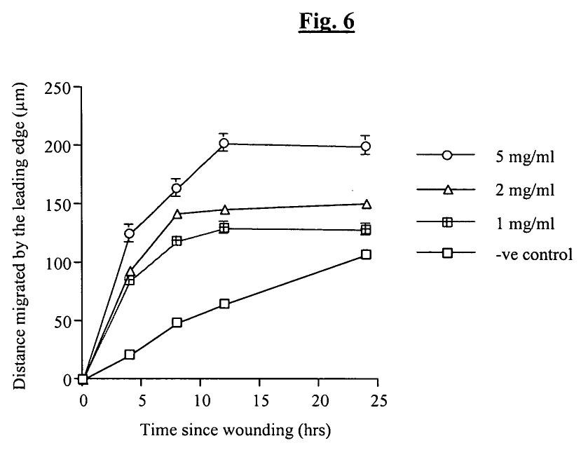

The dose response for the migratory distanced traveled by wounded HT-29 cell monolayers incubated with 1 mg protein/ml, 2 mg protein/ml, and 5 mg protein/ml colostrum solutions is shown in FIG. 6. While all three colostrum solutions induced enhanced cell migration compared with the control sample, at 12 hours, the 5 mg protein/ml colostrum sample produced approximately 200 μm of movement by the anterior edge of the cell monolayer, while the 1 mg protein/ml colostrum sample produced approximately 130 μm of cell movement (compared with approximately 60 μm for the negative control sample), thereby demonstrating that increased concentrations of colostrum appear to have induced increased amounts of cell migration.

On the basis of these studies, we analyzed the effect of various potentially bioactive factors given alone and in combination on the rate of movement of these wounded cell monolayers. In order to make such experiments manageable, a certain number of parameters had to be fixed. We therefore chose the 8 hour time point as optimal for analysis.

### EXAMPLE V

### The Comparative Effect of Colostrum vs. Milk-Based Products on Cell Migration in Intestinal Cells

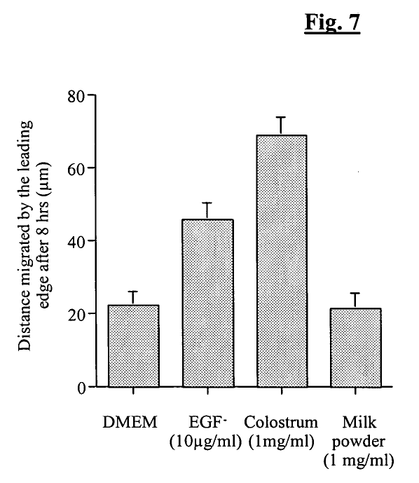

The next study analyzed the potential added benefit of using colostrum rather than milk based product. We therefore cultured HT-29 cells and wounded them as in accordance with the previously described Example IV protocol. Some wells acted as negative control (DMEM only), some contained EGF at 10 ug/ml (positive control), some contained colostrum (Sterling Technology) at 1 mg/ml, and some contained a similarly prepared skimmed milk product added at a final concentration of 1 mg protein/ml (Marvel™, Premier Brands UK limited, Spalding, Lincs, UK). The distance migrated by the leading edge of the cells 8 hours after wounding was then assessed. The results are shown in FIG. 7. As can be seen, the positive control (EGF) caused a two-fold increase in the amount of migration, the colostrum caused a three-fold increase, but the milk product was ineffective, causing no greater movement that that seen in cells grown in DMEM medium alone. Colostrum was therefore able to increase the rate of movement by about three-fold whereas the milk protein was ineffective.

### EXAMPLE VI

### Effect of Licorice on Cell Migration

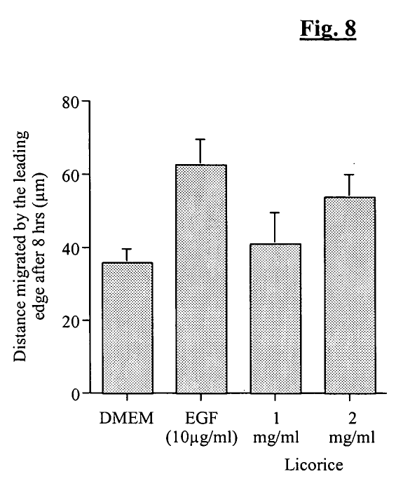

The next study analysed the potential added benefit of using licorice as a stimulant of restitution. We therefore cultured HT29 cells and wounded them in accordance with the Example IV protocol. Some wells acted as negative control (DMEM only), some had EGF (10 ug protein/ml) added as a positive control, and some had licorice extract (Whole Herb Company) added to the DMEM at either 1 or 2 mg/ml. The distance migrated by the leading edge of the cells 8 hours after wounding was then assessed. The results are shown in FIG. 8.

As can be seen, the positive control (EGF) increased the amount of cell movement, and the licorice was also seen to do this in a dose dependant manner.

### EXAMPLE VII

### Effect of Combining Colostrum and Soy on Cell Migration

In the next series of studies, we examined whether combining these factors may have added benefit to cell migration. Whenever potential effects of adding a combination of bioactive products together was being tested, we used the colostrum at a final concentration of 1 mg protein/ml, as our early studies had shown this to be an effective dose but below the maximal stimulatory level (so that extra effects due to the combination could be seen).

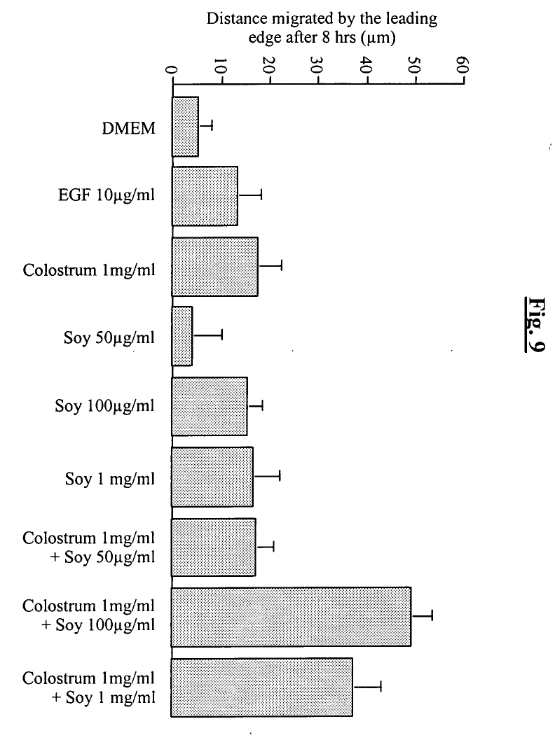

We therefore studied the amount of cell movement of HT29 cells that occurred 8 hours after wounding in wells that had DMEM alone (negative control), and wells that had also had added EGF 10 μg/ml (positive control), colostrum alone at 1 mg protein/ml, three different doses of soy given alone, and the three different doses of soya plus colostrum added at 1 mg/ml. The results are shown in FIG. 9.

As can be clearly seen, additive/synergistic effects were seen when soy and colostrum were both added together to the wounded cells and the optimum, in this assay system, was colostrum at 1 mg/ml and soy at 100 μg/ml, where the amount of restitution obtained was far greater than that seen in the positive (EGF) control wells (column 2).

### EXAMPLE VIII

### Effect of the Various Bioactive Products, Alone and in Combination, on the Degree of Stomach Injury in a Rat Gastric Damaging Model

Cell culture studies provide valuable information regarding potential bioactivity. However, it is also useful to progress, where possible to the in vivo (living) situation. The indomethacin-induced rat gastric damaging model is a robust, well-validated model for analyzing potential growth factors, and is well known in the art. A prior example of its use is described in Playford, R. J., Floyd, D. N., MacDonald, C. E. et al., “Bovine Colostrum Is a Health Food Supplement Which Prevents NSAID-Induced Gut Damage,” Gut 44: 653-58 (May 1999).

Adult, male, Sprague Dawley rats were used for all studies. Under light ether anaesthesia rats had a 2 ml gavage (i.e., oral administration) of test products.

These products consisted of saline alone (negative control, 3 rats used for this group), colostrum (5 mg protein/ml; 4 rats used for this group), soya protein (0.1 mg protein/ml; 4 rats used for this group) and the combination applied together (4 rats used for this group).

These products were all administered in a total volume of 2 ml and all contained 5% hydroxymethyl-cellulose that acts as a thickening agent to keep the test product inside the stomach for the duration of the study. Thirty minutes after the gavage, all animals were lightly anaesthatized again with ether, given an injection of the non-steroidal anti-inflammatory drug, indomethacin (20 mg/kg, injected subcutaneuosly), and placed in standard ‘Bullman’ restraint cages for 3 hours.

At the end of this period, the animals were killed and their stomachs removed and inflated with 4 ml of 10% formalin. The next day the stomachs were opened and placed in fresh formalin prior to assessment. The stomachs were randomly coded and all analyses of gastric damage were assessed blind. Total ulcerated area (mm²/stomach) was assessed using a dissecting microscope (×10) with the aid of a square grid.

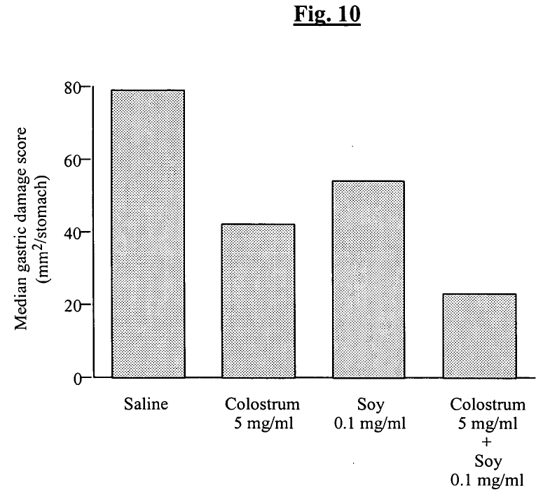

The results are shown in FIG. 10. As can be seen, negative control rats given only saline gavage had quite extensive ulceration (median 79 mm²/stomach). The pre-administration of colostrum reduced the amount of injury to 42 mm²/stomach, and the pre-administration of the soy reduced the amount of injury to 54 mm²/stomach. However, there was a much more profound reduction of injury in rats that had been given the combination of colostrum and soya, where the medium ulcer damage was only 23 mm²/stomach.

### EXAMPLE IX

### Effect of the Sodium Bicarbonate on the Degree of Stomach Injury in a Rat Gastric Damaging Model

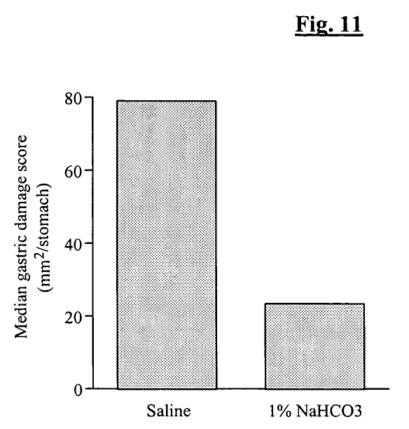

To examine the potential benefit of buffering the gastric acid, animals were treated as in Example VIII, except some animals received the saline gavage (negative control), and the others received 1% sodium bicarbonate via the gavage, instead of the colostrum or soy product. These results are shown in FIG. 11. As can be seen, compared to saline control, the degree of injury was markedly reduced by about 75% by the pre-administration of the sodium bicarbonate.

The above specification, examples and data provide a complete description of the manufacture and use of the composition of the invention. Since many embodiments of the invention can be made without departing from the spirit and scope of the invention, the invention resides in the claims hereinafter appended.

We claim:

1. A method of using a medicament comprising an enhanced bioactive agent composition for the prophylactic treatment of a gastrointestinal disorder in a mammal, wherein the enhanced bioactive agent composition comprises a bioactive agent and at least one bioactivity enhancing additive selected from the group consisting of a soybean product, licorice product, and sodium bicarbonate.

2. The method according to claim 1, wherein the bioactive agent comprises colostrum.

3. The method according to claim 2, wherein the colostrum is obtained from a cow.

4. The method according to claim 1, wherein the bioactive agent comprises lactoferrin, casein, or whey that has been extracted from colostrum or milk, or produced from plants or bacteria.

5. The method according to claim 1, wherein the bioactive agent has sufficient bioactivity, wherein when it is added to an intestinal cell in a culture medium at a final concentration of 1 mg protein/ml, its ability to stimulate growth (proliferation) of intestinal cells is at least 10% above the baseline level of growth for a corresponding intestinal cell in a culture medium without the bioactive agent or any other pro-stimulant, or its ability to stimulate movement (restitution) of such an intestinal cell in a culture medium is at least 10% above the baseline level of movement for a corresponding intestinal cell in a culture medium without the bioactive agent or any other pro-stimulant.

6. The method according to claim 3, wherein the colostrum is obtained from the cow within the first 72 hours post parturition.

7. The method according to claim 3, wherein the colostrum is obtained from the cow within the first 48 hours post parturition.

8. The method according to claim 3, wherein the colostrum is obtained from the cow within the first 24 hours post parturition.

9. The method according to claim 3, wherein the colostrum is obtained from the cow within the first two milkings following parturition.

10. The method according to claim 1, wherein the bioactive agent comprises spray-dried colostrum.

11. The method according to claim 1, wherein the soy product is selected from the group consisting of soy flour, soy protein isolate, soy protein concentrate, isolated soy protein, soy milk, hydrolyzed soy proteins, soybean oil, lecithin, defatted soy flakes, tofu, trypsin inhibitor, genistein, isoflavone, lectins, or other soybean subcomponent.

12. The method according to claim 11, wherein the soy flour, soy milk, hydrolyzed soy protein, defatted soy flakes, or tofu comprises about 2-25% by weight of the enhanced bioactive agent composition.

13. The method according to claim 11, wherein the soy protein isolate, protein concentrate, or isolated soy protein comprises about 2-20% by weight of the enhanced bioactive agent composition.

14. The method according to claim 11, wherein the trypsin inhibitor, genistein, isoflavone, lectin, or other soybean subcomponent comprises about 0.005-5.0% by weight of the enhanced bioactive agent composition.

15. The method according to claim 1, wherein the licorice product is selected from the group consisting of natural licorice root extract, DGL, and any hydrolized or ester amide derivative thereof.

16. The method according to claim 1, wherein the licorice product comprises about 0.5-25% by weight of the enhanced bioactive agent composition.

17. The method according to claim 1, wherein the sodium bicarbonate comprises about 0.5-25% by weight of the enhanced bioactive agent composition.

18. The method according to claim 10, wherein 330 mg -300 g of spray-dried enhanced bioactive agent composition is to be administered daily.

19. The method according to claim 18, wherein 330 mg-300 g of spray-dried enhanced bioactive agent composition is to be administered daily for the treatment of the mammal suffering from gastrointestinal disorder symptoms.

20. The method according to claim 18, wherein 330 mg-150 g of spray-dried enhanced bioactive agent composition is to be administered daily for the prevention of the onset of gastrointestinal disorder symptoms in the mammal.

21. The method according to claim 1, wherein the enhanced bioactive agent composition is in liquid form.

22. The method according to claim 21, wherein 1 -200 ml of liquid enhanced bioactive agent composition is to be administered daily.

23. The method according to claim 22, wherein 5 -200 ml of liquid enhanced bioactive agent composition is to be administered daily for the treatment of the mammal suffering from gastrointestinal disorder symptoms.

24. The method according to claim 22, wherein 2 -70 ml of liquid enhanced bioactive agent composition is to be administered daily for the prevention of the onset of gastrointestinal disorder symptoms in the mammal.

25. The method according to claim 1, wherein the gastrointestinal disorder is selected from the group consisting of apthous ulcers; esophagitis of the esophagus; gastritis or gastric ulcers in the stomach; duodenal ulcers, duodenitis, Crohn's Disease or Celiac Disease in the small intestine; ulcerative colitis, microscopic colitis, collagenous colitis, or diverticular disease in the large intestine; and non-ulcer dyspepsia in the stomach or small or large intestine.

26. The method according to claim 1, wherein the medicament further comprises hydroxymethyl cellulose, methyl cellulose, or guar gum.

27. The method according to claim 1, wherein the enhanced bioactive agent composition further comprises an added pure growth factor or immune modulator.

28. The method according to claim 27, wherein the pure growth factor or immune modulator is selected from the group consisting of EGF, transforming growth factor-α (“TGF-α”), transforming growth factor-β (“TGF-β”), pancreatic secretory trypsin inhibitor, glucagon-like peptide 2 (“GLP2”), or its analogues, growth hormone, GMCSF, hSP, interleukin-10, and lacoferrin.

29. The method according to claim 1, wherein the enhanced bioactive agent composition is administered by an oral route.

30. The method according to claim 29, wherein the enhanced bioactive agent composition is administered in the form of a capsule, tablet, liquid, food product, or drink product.

31. A method according to claim 1, wherein the enhanced bioactive agent composition is administered in the form of an enema.

32. A method of using a medicament comprising an enhanced bioactive agent composition for the prophylactic treatment of a skin ailment in a mammal, wherein the enhanced bioactive agent composition comprises a bioactive agent and a soy product.

33. The method according to claim 32, wherein the skin ailment is selected from the group consisting of viral-based cold sores, chickenpox, or shingles; a thermal, electrical, or chemical-induced burn to the skin; and cuts, wounds, or abrasions to the skin.

34. The method according to claim 32, wherein the enhanced bioactive agent composition further comprises a licorice product to assist in the penetration of the enhanced bioactive agent composition to prophylactically treat a virus-based skin ailment.

35. The method according to claim 32, wherein the enhanced bioactive agent composition further comprises a licorice product to assist in the transfer of the enhanced bioactive agent composition to a cut, wound, abrasion, or burn in the skin.

36. The method according to claim 32, wherein the bioactive agent comprises colostrum.

37. The method according to claim 36, wherein the colostrum is obtained from a cow.

38. The method according to claim 32, wherein the bioactive agent comprises lactoferrin, casein, or whey that has been extracted from colostrum or milk, or produced from plants or bacteria.

39. The method according to claim 32, wherein the bioactive agent has sufficient bioactivity, wherein when it is added to an intestinal cell in a culture medium at a final concentration of 1 mg protein/ml, its ability to stimulate growth (proliferation) of intestinal cells is at least 10% above the baseline level of growth for a corresponding intestinal cell in a culture medium without the bioactive agent or any other pro-stimulant, or its ability to stimulate movement (restitution) of such an intestinal cell in a culture medium is at least 10% above the baseline level of movement for a corresponding intestinal cell in a culture medium without the bioactive agent or any other pro-stimulant.

40. The method according to claim 37, wherein the colostrum is obtained from the cow within the first 72 hours post parturition.

41. The method according to claim 37, wherein the colostrum is obtained from the cow within the first 48 hours post parturition.

42. The method according to claim 37, wherein the colostrum is obtained from the cow within the first 24 hours post parturition.

43. The method according to claim 37, wherein the colostrum is obtained from the cow within the first two milkings following parturition.

44. The method according to claim 32, wherein the bioactive agent comprises spray-dried colostrum.

45. The method according to claim 32, wherein the soy product is selected from the group consisting of soy flour, soy protein isolate, soy protein concentrate, isolated soy protein, soy milk, hydrolyzed soy proteins, soybean oil, lecithin, defatted soy flakes, tofu, trypsin inhibitor, genistein, isoflavone, lectins, or other soybean subcomponent.

46. The method according to claim 45, wherein the soy flour, soy milk, hydrolyzed soy protein, defatted soy flakes, or tofu comprises about 2-25% by weight of the enhanced bioactive agent composition.

47. The method according to claim 45, wherein the soy protein isolate, protein concentrate, or isolated soy protein comprises about 2-20% by weight of the enhanced bioactive agent composition.

48. The method according to claim 45, wherein the trypsin inhibitor, genistein, isoflavone, lectin, or other soybean subcomponent comprises about 0.005-5.0% by weight of the enhanced bioactive agent composition.

49. The method according to claim 34, wherein the licorice product is selected from the group consisting of natural licorice root extract, DGL, and any hydrolized or ester amide derivative thereof.

50. The method according to claim 35, wherein the licorice product is selected from the group consisting of natural licorice root extract, DGL, and any hydrolized or ester amide derivative thereof.

51. The method according to claim 44, wherein 330 mg-150 g of spray-dried enhanced bioactive agent composition is to be administered daily.

52. The method according to claim 51, wherein 330 mg-150 g of spray-dried enhanced bioactive agent composition is to be administered daily for the treatment of the mammal suffering from skin ailment symptoms.

53. The method according to claim 51, wherein 330 mg-75 g of spray-dried enhanced bioactive agent composition is to be administered daily for the prevention of the onset of skin ailment symptoms in the mammal.

54. The method according to claim 32, wherein the enhanced bioactive agent composition is in liquid form.

55. The method according to claim 54, wherein 0.2 -20 ml of liquid enhanced bioactive agent composition is to be administered daily.

56. The method according to claim 55, wherein 0.5 -10 ml of liquid enhanced bioactive agent composition is to be administered daily for the treatment of the mammal suffering from skin ailment symptoms.

57. The method according to claim 55, wherein 0.5 -5 ml of liquid enhanced bioactive agent composition is to be administered daily for the prevention of the onset of skin ailment symptoms in the mammal.

58. The method according to claim 32, wherein the medicament further comprises hydroxymethyl cellulose, methyl cellulose, or guar gum.

59. The method according to claim 32, wherein the enhanced bioactive agent composition further comprises an added pure growth factor or immune modulator.

60. The method according to claim 59, wherein the pure growth factor or immune modulator is selected from the group consisting of EGF, transforming growth factor-α (“TGF-α”), transforming growth factor-β (“TGF-β”), pancreatic secretory trypsin inhibitor, glucagon-like peptide 2 (“GLP2”), or its analogues, growth hormone, GMCSF, hSP, interleukin-10, and lacoferrin.

61. The method according to claim 32, wherein the enhanced bioactive agent composition is administered by an oral route.

62. The method according to claim 61, wherein the enhanced bioactive agent composition is administered in the form of a capsule, tablet, liquid, food product, or drink product.

63. The method according to claim 32, wherein the enhanced bioactive agent composition is administered in the form of an enema.

64. A medicament composition comprising an enhanced bioactive agent composition for the prophylactic treatment of a gastrointestinal disorder in a mammal, wherein the enhanced bioactive agent composition comprises a bioactive agent and at least one bioactivity enhancing additive selected from the group consisting of a soybean product, licorice product, and sodium bicarbonate.

65. The medicament composition according to claim 64, wherein the bioactive agent comprises colostrum.

66. The medicament composition according to claim 65, wherein the colostrum is obtained from a cow.

67. The medicament composition according to claim 64, wherein the bioactive agent comprises lactoferrin, casein, or whey that has been extracted from colostrum or milk, or produced from plants or bacteria.

68. The medicament composition according to claim 64, wherein the bioactive agent has sufficient bioactivity, wherein when it is added to an intestinal cell in a culture medium at a final concentration of 1 mg protein/ml, its ability to stimulate growth (proliferation) of intestinal cells is at least 10% above the baseline level of growth for a corresponding intestinal cell in a culture medium without the bioactive agent or any other pro- stimulant, or its ability to stimulate movement (restitution) of such an intestinal cell in a culture medium is at least 10% above the baseline level of movement for a corresponding intestinal cell in a culture medium without the bioactive agent or any other pro-stimulant.

69. The method according to claim 64, wherein the soy product is selected from the group consisting of soy flour, soy protein isolate, soy protein concentrate, isolated soy protein, soy milk, hydrolyzed soy proteins, soybean oil, lecithin, defatted soy flakes, tofu, trypsin inhibitor, genistein, isoflavone, lectins, or other soybean sub component.

70. The method according to claim 69, wherein the soy flour, soy milk, hydrolyzed soy protein, defatted soy flakes, or tofu comprises about 2-25% by weight of the enhanced bioactive agent composition.

71. The method according to claim 69, wherein the soy protein isolate, protein concentrate, or isolated soy protein comprises about 2-20% by weight of the enhanced bioactive agent composition.

72. The method according to claim 69, wherein the trypsin inhibitor, genistein, isoflavone, lectin, or other soybean subcomponent comprises about 0.005-5.0% by weight of the enhanced bioactive agent composition.

73. The method according to claim 64, wherein the licorice product is selected from the group consisting of natural licorice root extract, DGL, and any hydrolized or ester amide derivative thereof.

74. The method according to claim 64, wherein the licorice product comprises about 1-25% by weight of the enhanced bioactive agent composition.

75. The method according to claim 64, wherein the sodium bicarbonate comprises about 1-25% by weight of the enhanced bioactive agent composition.

76. A medicament composition comprising an enhanced bioactive agent composition for the prophylactic treatment of a skin ailment in a mammal, wherein the enhanced bioactive agent composition comprises a bioactive agent and a soybean product.

77. The medicament composition according to claim 76, wherein the bioactive agent comprises colostrum.

78. The medicament composition according to claim 76, wherein the colostrum is obtained from a cow.

79. The medicament composition according to claim 76, wherein the bioactive agent comprises lactoferrin, casein, or whey that has been extracted from colostrum or milk, or produced from plants or bacteria.

80. The medicament composition according to claim 76, wherein the bioactive agent has sufficient bioactivity, wherein when it is added to an intestinal cell in a culture medium at a final concentration of 1 mg protein/ml, its ability to stimulate growth (proliferation) of intestinal cells is at least 10% above the baseline level of growth for a corresponding intestinal cell in a culture medium without the bioactive agent or any other pro- stimulant, or its ability to stimulate movement (restitution) of such an intestinal cell in a culture medium is at least 10% above the baseline level of movement for a corresponding intestinal cell in a culture medium without the bioactive agent or any other pro-stimulant.

81. The method according to claim 76, wherein the soy product is selected from the group consisting of soy flour, soy protein isolate, soy protein concentrate, isolated soy protein, soy milk, hydrolyzed soy proteins, soybean oil, lecithin, defatted soy flakes, tofu, trypsin inhibitor, genistein, isoflavone, lectins, or other soybean sub component.

82. The method according to claim 81, wherein the soy flour, soy milk, hydrolyzed soy protein, defatted soy flakes, or tofu comprises about 2-25% by weight of the enhanced bioactive agent composition.

83. The method according to claim 81, wherein the soy protein isolate, protein concentrate, or isolated soy protein comprises about 2-20% by weight of the enhanced bioactive agent composition.

84. The method according to claim 81, wherein the trypsin inhibitor, genistein, isoflavone, lectin, or other soybean subcomponent comprises about 0.005-5.0% by weight of the enhanced bioactive agent composition.

85. The method according to claim 76, wherein the licorice product is selected from the group consisting of natural licorice root extract, DGL, and any hydrolized or ester amide derivative thereof.

86. The method according to claim 85, wherein the licorice product comprises about 1-25% by weight of the enhanced bioactive agent composition.

87. The method according to claim 76, wherein the sodium bicarbonate comprises about 1-25% by weight of the enhanced bioactive agent composition.
